(19) United States
(12) Patent Application Publication
(10) Pub. No.: US 2026/0241069 A1
(43) Pub. Date: Aug. 20, 2026

(54) Method for Preparing a Sterile Hydrogel Comprising a Cross-Linked or Non-Crosslinked Polysaccharide or a Mixture Thereof

(71) Applicant: TEOXANE SA, GENEVE (CH)

(72) Inventors: Romain BRUSINI, VALLEIRY (FR); Camille VANTOU, ECHENEVEX (FR); Jimmy FAIVRE, VALSERHONE (FR); François BOURDON, GAILLARD (FR)

(21) Appl. No.: 19/164,392

(22) PCT Filed: Mar. 21, 2024
(86) PCT No.: PCT/EP2024/057635

§ 371 Date: Sep. 11, 2025

(30) Foreign Application Priority Data
Mar. 21, 2023 (FR) 2302635

(51) Int. Cl. A61L 27/20; A61L 27/52; C08B 37/08; C08J 3/24; C08K 5/092; C08K 5/098

(52) U.S. Cl. CPC A61L 27/20; A61L 27/52; C08B 37/0072; C08J 3/24; C08K 5/092; C08K 5/098

(57) ABSTRACT

The disclosure relates to a method for preparing a sterile hydrogel comprising a cross-linked polysaccharide, a non-cross-linked polysaccharide, or a mixture thereof, the method comprising the following steps:
(1) preparing a hydrogel comprising a cross-linked polysaccharide, a non-cross-linked polysaccharide or mixture thereof and further comprising at least 1 mM citrate ions; and
(2) sterilizing, preferably by heat, the hydrogel comprising at least 1 mM citrate ions to obtain a sterile hydrogel comprising a cross-linked polysaccharide, a non-cross-linked polysaccharide or a mixture thereof.

### FIELD OF THE INVENTION

The present invention concerns a method for preparing a sterile hydrogel comprising a cross-linked polysaccharide, a non-cross-linked polysaccharide or a mixture thereof, in particular comprising a cross-linked hyaluronic acid, a non-cross-linked hyaluronic acid or a mixture thereof.

### Technological Background

Polysaccharides, such as glycosaminoglycans, are widely used in the medical and esthetic fields, especially for filling soft tissues. In particular, the majority of products marketed for esthetic applications are based on hyaluronic acid. To improve skin quality, hydrogels prepared from unmodified hyaluronic acid are interesting because they have the advantage of being perfectly biocompatible.

It is also possible to use hydrogels based on modified hyaluronic acid, hyaluronic acid being usually modified by cross-linking. This cross-linking has the advantage of increasing the in vivo durability and in vivo resistance to degradation of hydrogels. Hydrogels based on cross-linked hyaluronic acid can be obtained by various preparation methods.

In addition, hydrogels based on cross-linked and/or non-cross-linked hyaluronic acid intended for soft tissue filling must be sterile. Thus, the methods for preparing hydrogels based on cross-linked and/or non-cross-linked hyaluronic acid intended to be injected generally comprise a step of sterilizing the hydrogel previously formed. Sterilization is typically carried out by heat, for example in an autoclave. It has been observed that these sterilization conditions tend to degrade cross-linked and/or non-cross-linked hyaluronic acid, leading to degradation of the rheological properties of the hydrogels.

Thus, a need remains for the provision of a method for the preparation of sterile hydrogels comprising a cross-linked (e.g. cross-linked hyaluronic acid) and/or non-cross-linked (e.g. non-cross-linked hyaluronic acid) polysaccharide which is as respectful as possible of the properties of hydrogels, i.e., which causes the least possible degradation of the rheological properties of hydrogels during sterilization, for example by heat, as well as over time.

### BRIEF DESCRIPTION OF THE INVENTION

The present invention relates to a method for preparing a sterile hydrogel comprising a cross-linked polysaccharide, a non-cross-linked polysaccharide, or a mixture thereof, the method comprising the following steps:
(1) preparing a hydrogel comprising a cross-linked polysaccharide, a non-cross-linked polysaccharide or mixture thereof and further comprising at least 1 mM citrate ions; and
(2) sterilizing, preferably by heat, the hydrogel to obtain a sterile hydrogel comprising a cross-linked polysaccharide, a non-cross-linked polysaccharide or a mixture thereof.

The invention also relates to a hydrogel obtained by the method of the invention. Finally, the invention relates to the use of citrate ions to protect a hydrogel comprising a cross-linked polysaccharide, a non-cross-linked polysaccharide or mixture thereof, in particular a cross-linked, non-cross-linked hyaluronic acid or mixture thereof, and optionally an anesthetic agent, from degradation of its rheological properties during sterilization, preferably by heat, and to the use of citrate ions to preserve the stability over time of hydrogels comprising a cross-linked polysaccharide, a non-cross-linked polysaccharide, or a mixture thereof, in particular a cross-linked hyaluronic acid, a non-cross-linked hyaluronic acid or mixture thereof, and optionally an anesthetic agent. Other aspects of the invention are as described below and in the claims.

### DETAILED DESCRIPTION OF THE INVENTION

### Definitions

The term “gel” designates a network of polymers which is expanded throughout its volume by a fluid. This means that a gel consists of two media, one solid and the other liquid, dispersed in one another. The so-called solid medium is composed of long polymer molecules connected together by weak bonds (for example hydrogen bonds) or by covalent bonds (cross-linking). The liquid medium is composed of a solvent. A gel generally corresponds to a viscoelastic product which has a phase angle δ of less than or equal to 90°, preferably less than or equal to 70°, preferably less than or equal to 45°, at 1 Hz for a deformation of 0.1% or a pressure of 1 Pa, advantageously a phase angle δ ranging from 2° to 45° or ranging from 20° to 45°.

The term “hydrogel” designates a gel as defined above in which the solvent constituting the liquid medium is predominantly water (for example at least 90%, in particular at least 95%, especially at least 97%, especially at least 98% by weight of the liquid medium) and has a pH ranging from 6.8 to 7.8.

The term “injectable hydrogel” designates a hydrogel that can flow and be injected manually by means of a syringe equipped with a needle of diameter ranging from 0.1 to 0.5 mm, for example a hypodermic needle of 32 G, 30 G, 27 G, 26 G or 25 G. Preferentially, an injectable hydrogel is a hydrogel having an average extrusion force of less than or equal to 25 N, preferably ranging from 5 to 25 N, more preferably ranging from 8 to 15 N, when measured with a dynamometer, at a fixed speed of approximately 12.5 mm/min, in syringes of external diameter greater than or equal to 6.3 mm, with a needle of external diameter less than or equal to 0.4 mm (27 G) and length of ½ inch, at room temperature.

A “superficial application” designates the administration, for example by mesotherapy, of a composition superficially into the skin, or onto the skin, for the treatment of the superficial layers of the skin, the epidermis and the most superficial parts of the dermis, to reduce superficial wrinkles and/or to improve the quality of the skin (such as its radiance, density or structure) and/or to rejuvenate the skin.

A “middle application” designates the administration of a composition to the middle part of the skin to treat the middle layers of the skin, as well as to reduce middle wrinkles. A “deep application” designates the administration of a composition into the deepest layers of the skin, the hypodermis and the deepest part of the dermis, and/or under the skin (above the periosteum) to add volume, such as for filling the deepest wrinkles and/or partially atrophied areas of the contour of the face and/or body. The so-called volumizing hydrogels can typically be administered for deep application.

A “cross-linked polysaccharide” designates a polysaccharide modified during a cross-linking reaction.

On the contrary, a “non-cross-linked polysaccharide” designates a polysaccharide not modified with a cross-linking agent and which has therefore not undergone a cross-linking reaction.

The term “cross-linking agent” designates any compound capable of introducing cross-linking between different polysaccharide chains.

The “molar cross-linking rate” (CR), expressed in %, designates the molar ratio of the quantity of cross-linking agent relative to the quantity of repeating unit of the polysaccharide introduced into the cross-linking reaction medium expressed per 100 moles of repeating units of the polysaccharide in the cross-linking medium. For example, a molar cross-linking rate of 1% means that there is one molecule of cross-linking agent introduced into the reaction medium per 100 moles of polysaccharide repeating units. The expression “repeating unit” of a polysaccharide designates a structural unit composed of one or more (usually 1 or 2) monosaccharides whose repetition produces the complete polysaccharide chain.

The “degree of modification” (MOD) of a polysaccharide, such as hyaluronic acid, is the molar quantity of cross-linking agent bound to the polysaccharide by one or more of its ends, expressed per 100 moles of repeating units of the polysaccharide. It can be determined by methods known to the person skilled in the art, such as nuclear magnetic resonance spectroscopy (NMR). For example, a degree of modification of 1% means that there is one molecule of cross-linking agent per 100 moles of polysaccharide repeating units.

The term “polysaccharide” designates a polymer composed of monosaccharides (preferentially D enantiomers) joined together by glycosidic bonds.

The term “room temperature” is understood to mean a temperature ranging from 20 to 25° C., more particularly 21° C.

The linear viscoelastic region (LVER) corresponds to the range of deformations of the hydrogel from an initial value of elastic modulus G′ to the value of elastic modulus G′ minus 10% of its initial value. The LVER measurement consists of an oscillatory strain sweep measurement in compression mode at a given oscillation frequency to determine the linear viscoelastic region.

### Method

Unexpectedly, the inventors have discovered that the addition of citrate ions during the preparation of hydrogels comprising a cross-linked and/or non-cross-linked polysaccharide, in particular a cross-linked and/or non-cross-linked hyaluronic acid, makes it possible to effectively protect the hydrogel from degradation of its rheological properties during sterilization, in particular during sterilization by heat. The hydrogels obtained by the method of the present invention thus exhibit fewer changes in their rheological properties compared to hydrogels prepared by an equivalent method without addition of citrate ions. The hydrogels obtained by the method of the present invention also exhibit better preservation of their rheological properties over time.

The present invention thus relates to a method for preparing a sterile hydrogel comprising a cross-linked and/or non-cross-linked polysaccharide, the method comprising the following steps:
(1) preparing a hydrogel comprising a cross-linked and/or non-cross-linked polysaccharide and further comprising at least 1 mM citrate ions; and
(2) sterilizing, preferably by heat, the hydrogel to obtain a sterile hydrogel comprising a cross-linked and/or non-cross-linked polysaccharide.

The hydrogel comprising a cross-linked and/or non-cross-linked polysaccharide and additionally comprising at least 1 mM citrate ions according to step (1) can be prepared according to two alternative methods:
method 1: by adding citrate ions in powder form or as a solution during the preparation of a hydrogel from a previously cross-linked and/or non-cross-linked polysaccharide; or
method 2: when the hydrogel comprises a cross-linked polysaccharide, by carrying out the cross-linking of the polysaccharide in a reaction medium comprising citrate ions and then preparing the hydrogel from the cross-linked polysaccharide obtained.

### Method 1

When the method of the present invention implements method 1, step (1) of preparing the hydrogel comprises a step of adding, to the cross-linked polysaccharide or to the non-cross-linked polysaccharide or to their mixture, a solution comprising citrate ions in a quantity sufficient to obtain a citrate ion concentration of at least 1 mM in the hydrogel. In a variant, when the method of the present invention implements method 1, step (1) of preparing a hydrogel comprises a step of adding, to the cross-linked polysaccharide or to the non-cross-linked polysaccharide or to their mixture, citrate ions in powder form in a quantity sufficient to obtain a citrate ion concentration of at least 1 mM in the hydrogel.

In some embodiments, the preparation of the hydrogel comprises the addition of citrate ions in powder form and in solution form, preferably the powder and solution being added at different stages of the preparation of the hydrogel.

The Cross-Linked and/or Non-Cross-Linked Polysaccharide

The polysaccharide can be any polymer composed of monosaccharides joined together by glycosidic bonds or mixtures thereof. Preferably, the polysaccharide is chosen from pectin and pectic substances; chitosan; chitin; cellulose and its derivatives; agarose; glycosaminoglycans such as hyaluronic acid, heparosan, dermatan sulfate, keratan sulfate, chondroitin and chondroitin sulfate; and mixtures thereof. Still more preferably, the polysaccharide is chosen from hyaluronic acid, heparosan, chondroitin and mixtures thereof, still more preferentially, the polysaccharide is hyaluronic acid or one of its salts, in particular in a physiologically acceptable salt such as sodium salt, potassium salt, zinc salt, calcium salt, magnesium salt, silver salt, calcium salt and mixtures thereof. More particularly, hyaluronic acid is in acid form or in sodium salt form (NaHA). The hydrogel can thus be a hydrogel based on hyaluronic acid and/or one of its salts.

Preferably, if the polysaccharide is hyaluronic acid, it has a weight average molecular weight (Mw) ranging from 0.05 to 10 MDa, preferentially ranging from 0.5 to 5 MDa, for example ranging from 2 to 4 MDa or ranging from 1 to 5 MDa.

The polysaccharide may be provided in hydrated form (totally or partially hydrated) or in dry form, such as in powder or fiber form. When the polysaccharide is supplied in hydrated form, it is typically in gel form.

A cross-linked polysaccharide can be prepared by any method known to the person skilled in the art.

The cross-linked polysaccharide may result from the reaction of the polysaccharide with a cross-linking agent or from the reaction of a modified polysaccharide to allow the formation of covalent intermolecular bonds.

For example, the cross-linked polysaccharide may be prepared as described in WO 2010131175A1 and WO 201277054A1.

The method of the present invention may thus comprise, before the hydrogel preparation step, a step of preparing a cross-linked polysaccharide.

The cross-linked polysaccharide is preferably a cross-linked polysaccharide whose molar degree of cross-linking is less than or equal to 10%. Preferentially, the cross-linked polysaccharide is a cross-linked polysaccharide whose molar degree of cross-linking is greater than 0 and less than or equal to 6%. Even more preferentially, the cross-linked polysaccharide is a cross-linked polysaccharide whose molar degree of cross-linking is greater than 0 and less than or equal to 4%. Even more preferably, the cross-linked polysaccharide is a cross-linked polysaccharide whose molar degree of cross-linking is greater than 0 and less than or equal to 2%, preferably less than or equal to 1%, even more preferentially less than or equal to 0.8%, especially ranging from 0.1% to 0.5% (number of moles of cross-linking agent(s) per 100 moles of repeating unit of the polysaccharide(s)).

The polysaccharide may be cross-linked by reacting a polysaccharide that has been previously modified. The polysaccharide may have been modified by introducing functional groups capable of reacting with each other and forming covalent intermolecular bonds. The polysaccharide may have been modified by grafting with a molecule allowing subsequent cross-linking of the polysaccharide thus modified. For example, the polysaccharide may have been modified by grafting a silylated molecule, amino acid, amino acid derivative or protein.

The polysaccharide may be cross-linked by means of a cross-linking agent. The polysaccharide is preferably cross-linked by means of a cross-linking agent chosen from epoxide or non-epoxide bi- or multi-functional cross-linking agents, i.e., prepared by reaction of the polysaccharide with a cross-linking agent. Among the epoxide agents that may be mentioned are 1,4-butanediol diglycidyl ether (BDDE), 1,2,7,8-diepoxyoctane, 1,2-bis(2,3-epoxypropyl)-2,3-ethane (EGDGE), poly(ethylene glycol)-diglycidyl ether (PEGDE), and mixtures thereof. Among the non-epoxide agents that may be mentioned are endogenous polyamines such as spermine, spermidine and putrescine, aldehydes such as glutaraldehyde, carbodiimides and divinyl sulfone, hydrazide derivatives such as adipic acid dihydrazide, bisalkoxyamines, dithiols such as polyethylene glycol dithiol and mixtures thereof. Among the non-epoxide agents which may be mentioned are amino acids such as cysteine and lysine; peptides or proteins containing amino acids such as cysteine and lysine; poly(dimethyl siloxane); trimetaphosphates, such as, for example, sodium trimetaphosphate, calcium trimetaphosphate or barium trimetaphosphate.

In some embodiments, the cross-linking agent is an epoxy agent, preferably 1,4-butanediol diglycidyl ether (BDDE) or polyethylene glycol diglycidyl ether. Preferentially, the cross-linking agent is 1,4-butanediol diglycidyl ether (BDDE).

In some embodiments, the cross-linking agent is a non-epoxide agent, preferably chosen from endogenous polyamines, aldehydes, carbodiimides, divinyl sulfone, amino acids, peptides and mixtures thereof.

The cross-linked polysaccharide is preferably a cross-linked polysaccharide having a degree of modification (MOD) of less than or equal to 10%, preferably less than or equal to 6%, preferably less than or equal to 4%, preferably less than or equal to 2%, more preferably less than or equal to 1%. Advantageously, the cross-linked polysaccharide is a cross-linked polysaccharide having a degree of modification (MOD) of less than or equal to 1.8%, more preferably less than or equal to 1.5%, preferentially less than or equal to 1.2%, even more preferentially less than 1%.

In particular, the cross-linked polysaccharide can be prepared by a method comprising the following steps:
(a1) preparing a cross-linking reaction medium comprising one or more polysaccharides, one or more cross-linking agents and a solvent; and
(a2) reacting the reaction medium to obtain a cross-linked polysaccharide.

The polysaccharide is as described above.

In step (a1), the polysaccharide is provided in dry form such as in powder or fiber form, or in the hydrated form. When the polysaccharide is supplied in hydrated form, it is in the form of a non-cross-linked gel or solution. In particular, when the polysaccharide is in hydrated form, it is an aqueous non-cross-linked gel or aqueous solution.

The cross-linking agent is as described above.

The solvent is typically water or a mixture comprising water and an organic solvent (typically a mixture comprising at least 90% by weight of water, or at least 95% or at least 99% by weight of water relative to the total weight of the solvent). For example, an organic solvent such as an alcohol, particularly ethanol, or DMSO, can be used to solubilize the cross-linking agent, for example when it is poly(dimethylsiloxane) terminated at each end with a diglycidyl ether (CAS number: 130167-23-6), before its addition to the aqueous reaction medium.

The reaction medium may also comprise salts, pH adjusters, for example a Bronsted base, more preferentially a hydroxide salt, such as sodium or potassium hydroxide, additional components as described below and mixtures thereof. The addition of a Bronsted base can especially be necessary when the functional groups Z of the cross-linking agent have an epoxide group or a vinyl group. In these cases, cross-linking takes place at a pH greater than or equal to 10, more advantageously greater than or equal to 12, which requires the addition of a Bronsted base to the reaction medium, typically at a concentration comprised between 0.10 M and 0.30 M.

The total quantity of cross-linking agent in the reaction medium typically varies from 0.001 to 0.10 moles per 1 mole of polysaccharide repeating unit, preferably from 0.001 to 0.08 moles or from 0.001 to 0.06 moles per 1 mole of polysaccharide repeating unit, preferentially from 0.001 to 0.04 moles per 1 mole of polysaccharide repeating unit, preferably from 0.001 to 0.03 moles per 1 mole of polysaccharide repeating unit, preferably 0.001 to 0.02 moles per 1 mole of polysaccharide repeating unit, more preferably from 0.001 to 0.01 moles per 1 mole of polysaccharide repeating unit, even more preferably from 0.001 to 0.005 moles per 1 mole of polysaccharide repeating unit. When the polysaccharide is a glycosaminoglycan such as hyaluronic acid, the repeating unit is a disaccharide unit.

The concentration by mass of polysaccharide or polysaccharide salt in the reaction medium advantageously ranges from 50 to 300 mg/g of solvent, preferably from 80 to 200 mg/g.
Step (a1) typically comprises a step of homogenizing the reaction medium. Homogenization is generally carried out by three-dimensional stirring, stirring with a mixer, stirring with blades or stirring with a spatula.
Step (a1) is typically carried out at a temperature ranging from 4 to 35° C., preferably ranging from 15° C. to 25° C. Preferably, the duration of step (1) does not exceed 5 hours. It generally varies from 15 minutes to 4 hours, preferably from 30 minutes to 2 hours.
Step (a2) consists of reacting the reaction medium to obtain a cross-linked polysaccharide. Advantageously, step (a2) is carried out directly after step (a1).

This step makes it possible to cross-link the polysaccharide chains with one another. The functional groups of the cross-linking agent react with functional groups present on the polysaccharides to bind the polysaccharide chains with one another and to cross-link them by forming intermolecular bonds. The cross-linking agent can also react with functional groups present on the same polysaccharide molecule so as to form intramolecular bonds. In particular, the functional groups of the cross-linking agent react with the —OH or —COOH or —CHO groups present on polysaccharides such as hyaluronic acid. Cross-linked polysaccharides comprising at least one cross-linking link between two polysaccharide chains, said cross-linking link being the residue of the cross-linking agent, are thus obtained.

The cross-linking can be carried out in the presence of several cross-linking agents. When cross-linking is carried out in the presence of several cross-linking agents, the cross-linking agents can be added simultaneously or separately over time to the reaction medium. Step (a2) can thus comprise repeated cross-linking steps; advantageously step (a2) comprises a single cross-linking step. The cross-linking is then carried out in the presence of a total quantity of cross-linking agents typically ranging from 0.1 to 10 moles, or from 0.1 to 8 moles, or from 0.1 to 6 moles, or from 0.1 to 4 moles, or from 0.1 to 3 moles, or from 0.1 to 2 moles or from 0.1 to 1 mole or from 0.1 to 0.8 moles or from 0.1 to 0.5 moles of cross-linking agents (or their salts) per 100 moles of repeating unit of the polysaccharide. The cross-linking conditions, in particular the contents of cross-linking agent, duration and temperatures, as well as the weight average molecular weights (Mw) of the polysaccharide used are interdependent.

The lower the content of cross-linking agent, the longer the reaction time must be in order to obtain similar mechanical properties for the resulting cross-linked polysaccharide, and ultimately of the prepared hydrogel. In other words, the lower the molar percentage of cross-linking agent, the fewer reactive functional groups there are in the reaction medium and the lower the probability that 2 groups will meet and react together, the longer the reaction time must be in order to allow the functional groups to react with one another and form cross-linking bonds, and thus ultimately obtain a hydrogel with desirable properties.

In some embodiments, step (a2) can be carried out by placing the reaction medium directly obtained at the end of step (a1) at a temperature of less than or equal to 30° C., preferably less than or equal to 25° C. The temperature is typically greater than 0° C. or greater than 5° C. or even greater than 10° C. Even more preferably, step (a2) can be carried out by placing the reaction medium directly obtained at the end of step (a1) at a temperature equal to room temperature. When step (a2) is carried out at a temperature greater than or equal to 0° C. and less than or equal to 30° C., the cross-linking duration is at least 1 minute, preferably at least 10 minutes, even more preferably at least 1 hour. Preferably, the duration of cross-linking is at most 5 days.

In some embodiments, step (a2) can be carried out by placing the reaction medium obtained at the end of step (a1) at a temperature greater than 30° C., or greater than or equal to 35° C., or greater than or equal to 40° C., or greater than or equal to 45° C., or greater than or equal to 50° C. The temperature is typically less than 60° C. When the temperature is greater than 30° C., the duration of the cross-linking step is at least greater than or equal to 1 minute, preferably at least greater than or equal to 10 minutes, still more preferably at least 1 hour, preferably comprised between 1 hour and 5 hours. In some embodiments, step (a2) can be carried out by placing the reaction medium directly obtained at the end of step (a1) at a temperature ranging from 0 to 15° C. or from 1 to 10° C. or from 1 to 9° C.

In some embodiments, step (a2) can be carried out by placing the reaction medium obtained at the end of step (a1), at a pressure P less than or equal to atmospheric pressure and at a temperature T greater than the eutectic point temperature of the reaction medium as measured at pressure P and less than the freezing point temperature of the reaction medium as measured at pressure P, preferably for a period of at least 1 hour. Hydrogels based on cross-linked polysaccharide prepared by such a method are highly biocompatible. Indeed, cross-linked polysaccharides can be prepared with smaller quantities of cross-linking agent, for example quantities ranging from 0.001 to 0.02 moles per 1 mole of repeating unit of the polysaccharide.

The freezing point temperature of the reaction medium designates the temperature at which the mixture of the components of the reaction medium, on a macroscopic scale, solidifies, i.e., becomes non-fluid. Below the freezing point, the mixture is in a freezing state which is characterized by the coexistence of components in solid and liquid form. The freezing state is maintained up to the eutectic point temperature of the reaction medium.

The eutectic point temperature of the reaction medium refers to the temperature below which the mixture of the components of the reaction medium changes from a freezing state (coexistence of liquid and solid phases) to a completely solid state, i.e., a state in which all the components of the mixture are in solid form. The freezing point and eutectic point of a mixture depend on the pressure to which the mixture is subjected so the freezing point and eutectic point are measured at the pressure P.

The freezing point and eutectic point can be determined by differential scanning calorimetry. This method makes it possible to determine the phase transitions. To do this, the product to be studied is gradually cooled until its phase transitions are observed. The temperature T is preferably greater than or equal to −55° C. and less than or equal to −5° C., preferably it ranges from −35° C. to −10° C. More preferably still, the temperature T is approximately −20° C.

The pressure P is preferably atmospheric pressure. Atmospheric pressure is the pressure exerted by the air making up the atmosphere on any surface in contact with it. It varies according to altitude. At an altitude of 0 m, the average atmospheric pressure is 101,325 Pa. Preferably, the pressure P is atmospheric pressure and the temperature T is greater than or equal to −55° C. and less than or equal to −5° C.; preferably T varies from −35° C. to −10° C. or is approximately −20° C.

Preferably, during cross-linking step (a2), when the temperature T is greater than or equal to −55° C. and less than or equal to −5° C., the reaction medium obtained at the end of step (1) is placed for a period of at least 1 hour, preferably at least 3 hours, preferably at least 72 hours, preferably at most 27 weeks under these conditions. Preferably, the cross-linking step (a2) is carried out for a period ranging from 2 to 25 weeks, preferably ranging from 2 to 20 weeks or 2 to 17 weeks, even more preferably from 3 to 8 weeks or 4 to 7 weeks and at temperature T and pressure P.

At the end of step (a2), the cross-linked polysaccharide is typically in the form of a gel. This gel is generally directly used in the remainder of the method of the invention (step 1).

The cross-linked and/or non-cross-linked polysaccharides described above are useful for implementing the method of the invention and thus preparing hydrogels comprising a cross-linked and/or non-cross-linked polysaccharide. The cross-linked or non-cross-linked polysaccharide, or a mixture thereof, will constitute the polymer network of the hydrogel. The hydrogel comprising a cross-linked or non-cross-linked polysaccharide or a mixture thereof may thus be said to be based on a cross-linked polysaccharide or a non-cross-linked polysaccharide or a mixture thereof. A hydrogel comprising a non-cross-linked polysaccharide as the only polysaccharide is prepared from a non-cross-linked polysaccharide. A hydrogel comprising a cross-linked polysaccharide as the only polysaccharide is prepared from a cross-linked polysaccharide. When the hydrogel comprises a mixture of a cross-linked and non-cross-linked polysaccharide, the hydrogel is prepared from a cross-linked polysaccharide and a non-cross-linked polysaccharide. The non-cross-linked polysaccharide is typically added to the cross-linked polysaccharide during hydrogel preparation.

### Hydrogel Preparation (Step (1))

The method of the present invention according to method 1 comprises the preparation of a hydrogel comprising a cross-linked polysaccharide, a non-cross-linked polysaccharide or a mixture thereof and further comprising at least 1 mM of citrate ions, preferably from 1 to 12 mM of citrate ions.

The preparation of the hydrogel comprises at least one step of adding citrate ions to the cross-linked and/or non-cross-linked polysaccharide. The quantity of citrate ions added in this step makes it possible to obtain a concentration of citrate ions in the hydrogel prepared of at least 1 mM, preferably ranging from 1 to 12 mM.

The preparation of the hydrogel advantageously comprises a step of adjustment to the physiological pH, especially ranging from 6.8 to 7.8.

In a variant, citrate ions are added in powder form to the cross-linked and/or non-cross-linked polysaccharide. The quantity of citrate ions in powder form added in this step makes it possible to obtain a citrate ion concentration in the hydrogel prepared of at least 1 mM, preferably ranging from 1 to 12 mM. Typically when citrate ions are added in powder form, the effect of citrate ions on the pH of the hydrogel is neutralized.

In a variant, citrate ions are added as a solution (solution comprising citrate ions) to the cross-linked and/or non-cross-linked polysaccharide. The quantity of the solution comprising citrate ions added in this step makes it possible to obtain a concentration of citrate ions in the prepared hydrogel of at least 1 mM. Preferably, the concentration of citrate ions in the hydrogel varies from 1 to 150 mM or from 1 to 100 mM or from 1 to 50 mM or from 1 to 20 mM or from 1 to 12 mM.

In some embodiments, the quantity of citrate ions added (as a solution or powder) makes it possible to obtain a citrate ion concentration in the hydrogel of at least 1.5 mM, or at least 2 mM, or at least 2.5 mM, or at least 3 mM or at least 3.5 mM. The maximum concentration of citrate ions in the hydrogel is generally 12 mM.

In some embodiments, the quantity of citrate ions added (as a solution or powder) makes it possible to obtain a concentration of citrate ions in the hydrogel ranging from 2 to 12 mM, or from 3 to 11 mM, or from 3 to 9 mM, or from 3 to 8 mM, or from 4 to 8 mM or from 3 to 5 mM.

In some embodiments, the quantity of citrate ions added (as a solution or powder) makes it possible to obtain a concentration of citrate ions in the hydrogel ranging from 5 to 12 mM.

A solution comprising citrate ions means a solution whose pH allows citrate ions to be present in solution in this solution or means a solution capable of releasing citrate ions once added during the preparation of the hydrogel. The solution comprising citrate ions is preferably prepared from citric acid or sodium citrate, calcium citrate, potassium citrate or magnesium citrate.

The solution comprising added citrate ions typically has a pH ranging from 6.8 to 7.8 (physiological pH). If the solution does not have such a pH, the pH is adjusted, as necessary, during hydrogel preparation so that the final hydrogel has such a pH. The concentration of citrate ions in the solution is chosen so as to limit the dilution effect that can be generated by the addition of the solution during the preparation of the hydrogel, such a dilution effect of the hydrogel being undesirable. The maximum concentration of citrate ions that can be added to the hydrogel is then limited by adjusting the pH. Indeed, adjusting the pH of the solution to reach a physiological pH is less easy beyond a certain concentration of citrate ions. The solution comprising citrate ions is typically prepared so that the solution is concentrated in citrate ions, for example 100 times more concentrated compared to the final concentration of citrate ions in the hydrogel.

The solution comprising citrate ions is typically prepared in water or a physiologically acceptable buffer, preferably by adding citric acid or sodium citrate, or calcium citrate, or potassium citrate or magnesium citrate to water or a physiologically acceptable buffer. Examples of buffers include, but are not limited to, N-(carbamoylmethyl) taurine (CAS No.: 7365-82-4), the sodium salt of 3-[N, N-bis(hydroxyethyl)amino]-2-hydroxypropane sulfonic acid (CAS No: 102783-62-0), 3-morpholino-2-hydroxypropane sulfonic acid (CAS No: 68399-77-9), 1,4-piperazinediethanesulfonic acid (CAS No: 5625-37-6), 1,4-piperazine-N,N′-bis(propanesulfonic) acid (CAS No: 5625-56-9), 2-hydroxy-3-[tris(hydroxymethyl)methylamino]-1-propanesulfonic acid (CAS No: 68399-81-5), 2-[(2-hydroxy-1, 1-bis(hydroxymethyl)ethyl)amino]ethane sulfonic acid (CAS No: 7365-44-8), N-[tris(hydroxymethyl)methyl] glycine (CAS No: 5704 Apr. 1), 3-(N-morpholino) propanesulfonic acid (CAS No: 1132-61-2), tris(hydroxymethyl)aminomethane (CAS No: 77-86-1), bis(2-hydroxyethyl)amino-tris(hydroxymethyl) methane (CAS No: 6976-37-0), N, N-bis(2-hydroxyethyl) taurine (CAS No: 10191-18-1), 4-(2-hydroxyethyl) piperazine-1-ethanesulfonic acid (CAS No: 7365-45-9), 1,4-piperazinediethanesulfonic acid (CAS No: 5625-37-6), 4-(2-hydroxyethyl) piperazine-1-(2-hydroxypropane-3-sulfonic acid) (CAS No: 68399-78-0), phosphate buffers such as PBS with a pH around physiological pH (CAS No: 7647-14-5, 7447-40-7).

Preferably, the buffer is chosen from 3-(N-morpholino) propanesulfonic acid (CAS No: 1132-61-2), tris(hydroxymethyl)aminomethane (CAS No: 77-86-1), bis(2-hydroxyethyl)amino-tris(hydroxymethyl) methane (CAS No: 6976-37-0), N, N-bis(2-hydroxyethyl) taurine (CAS No: 10191-18-1), 4-(2-hydroxyethyl) piperazine-1-ethane sulfonic acid (CAS No: 7365-45-9) and phosphate buffers such as PBS with a pH around physiological pH (CAS No: 7647-14-5, 7447-40-7).

Preferentially, the buffer is a phosphate buffer, particularly a saline solution buffer of NaH₂PO₄/Na₂HPO₄ or of KH₂PO₄/K₂ HPO₄. The pH of the solution comprising citrate ions is typically adjusted by adding acid or base.

Thus, in some embodiments, the solution comprising citrate ions is a solution of citric acid in a phosphate buffer, the pH of which ranges from 6.8 to 7.8.

In some embodiments, the solution comprising citrate ions is a solution of sodium citrate in a phosphate buffer, the pH of which ranges from 6.8 to 7.8.

In preferred embodiments, citrate ions are added as a solution comprising citrate ions, the solution being as described above. Preferably, the solution is a solution of citric acid or a solution of sodium citrate or calcium citrate, or potassium citrate or magnesium citrate. The solution is preferably a solution of citric acid or sodium citrate or calcium citrate or potassium citrate or magnesium citrate in a physiologically acceptable buffer, such as a phosphate buffer.

The preparation of a hydrogel from a cross-linked and/or non-cross-linked polysaccharide can be carried out conventionally, with the difference that citrate ions are added during the preparation of the hydrogel. Thus, the preparation of a hydrogel comprising a cross-linked and/or non-cross-linked polysaccharide may comprise one or more of the following conventional steps:
pH adjustment (1);
Dilution (2);
Purification (3);
Addition of at least one additional component (4);
Extrusion (5).

These steps well known to the skilled person can be as described below. They may be at least partly concomitant.

The conventional steps can be carried out in the following sequential manner: optional adjustment of the pH (1), then optional dilution (2), then optional purification (3), then optional addition of an additional component (4), then optional extrusion (5). They can also be carried out in a different order. Advantageously, the extrusion step (5) is carried out last, when at least one of the other conventional steps is implemented. It can also be carried out several times and be inserted between the other conventional steps described.

For example, the conventional steps can be carried out sequentially as follows: (1), (2), (3), (4), (5); or (2), (1), (3), (4), (5); or (2) (1), (4), (5); or (2), (4), (5); or (1), (4), (5); or (2), (4), (3), (5); or (2), (4), (1), (5); or (2), (4), (5); or (4), (2), (1); or (4), (1), (2); or (2), (3), (4), (5); or (4), (2), (3), (5); or (2), (4), (1); or (1), (5), (3), (4); or (1), (5), (4); or (2), (4). Steps (2), (3), (4) and (5) can be concomitant. For example, the preparation of the hydrogel may comprise the following sequence: (2) and (4) are carried out concomitantly.

Citrate ions (in powder form or in solution) can be added at the time of, before or after any of these conventional steps.

In a variant, the citrate ions are added before the extrusion step (5) to obtain a homogeneous gel.

When a purification step (3) is implemented, the citrate ions can be added before or after the purification step (3), advantageously the citrate ions are added after the purification step (3). The addition of citrate ions after the purification step ensures better control of the citrate ion concentration in the prepared hydrogel.

Preferably, citrate ions are added between purification step (3) and extrusion step (5).

Citrate ions can be added after dilution step (2) or during dilution step (2), for example, citrate ions can be added to the aqueous dilution solvent.

Preferably, the citrate ions are added during the dilution step (2) and/or during the step (4) of adding at least one additional component, preferably during the step (4) of adding at least one additional component. In particular, in some embodiments, the addition of the solution comprising citrate ions is concomitant with the step (4) of adding at least one additional component. In particular, in some embodiments, the addition of the solution comprising citrate ions is concomitant with the addition of an anesthetic solution. In particular, in some embodiments, the addition of the solution comprising citrate ions is concomitant with the addition of a lubricating agent. In some embodiments, the solution comprising added citrate ions may comprise other components, in particular a lubricating agent, such as non-cross-linked hyaluronic acid, non-cross-linked heparosan, or a mixture thereof.

The steps of dilution (2), addition of at least one additional component (4) and addition of citrate ions may be concomitant.

The citrate ions can be added after the pH adjustment step (1). The citrate ions can be added between the pH adjustment step (1) and extrusion step (5) when these two steps are implemented.

### pH Adjustment (1)

The hydrogel preparation method may comprise a step of adjusting the pH of the hydrogel to reach the desired pH (pH 6.8-7.8).

### Dilution (2)

The method for preparing the hydrogel may comprise a step of diluting the cross-linked and/or non-cross-linked polysaccharide. The dilution step makes it possible to adjust the polysaccharide concentration in the prepared hydrogel. In particular, an aqueous solvent is added to the cross-linked and/or non-cross-linked polysaccharide, for example, a physiological saline solution, possibly buffered by the presence of salts, such as phosphate salts. More particularly, the aqueous solvent added has a pH around physiological pH (6.8-7.8). The polysaccharide concentration obtained following the dilution step advantageously varies from 1 mg/g to 50 mg/g of hydrogel, more advantageously from 5 mg/g to 35 mg/g of hydrogel, even more advantageously from 10 mg/g to 30 mg/g of hydrogel.

### Purification (3)

The preparation method according to the invention may comprise at least one purification step.

The purification step aims to remove any undesirable impurities. These impurities may result from the cross-linking of the polysaccharide, for example resulting from step (a2) described above. Such impurities may comprise, for example, the residual cross-linking agent, in particular of the epoxide type, which would not have reacted.

This step can also be used to perform a liquid exchange, for example a buffer exchange.

The purification step can therefore be implemented most particularly when the hydrogel comprises a cross-linked polysaccharide.

Purification can be carried out by dialysis or else by filtration, for example by dynamic cross-flow filtration (DCF).

### Addition of Additional Components (4)

The method for preparing the hydrogel may comprise one or more steps of adding at least one additional component. The additional component may be chosen from anesthetics, antioxidants, lubricants, amino acids, peptides, proteins such as collagen and silk fibroin, vitamins, elements such as silicon (for example via the addition of orthosilicic acid), minerals, nucleic acids, nucleotides or polynucleotides such as PDRN, nucleosides, coenzymes, adrenergic derivatives, sodium dihydrogen phosphate monohydrate and/or dihydrate, sodium chloride and a mixture thereof.

Non-cross-linked polysaccharides, in particular non-cross-linked hyaluronic acid, non-cross-linked heparosan or mixtures thereof can be mentioned as examples of lubricating agents.

Examples of anesthetics include, in a non-limiting manner, ambucaine, amoxecaine, amylocaine, aprindine, aptocaine, articaine, benzocaine, betoxycaine, bupivacaine, butacaine, butamben, butanilicaine, chlorobutanol, chloroprocaine, cinchocaine, cocaine, cryofluorane, cyclomethycaine, dexivacaine, diamocaine, clodacaine, diperodon, dyclonine, etidocaine, euprocin, febuverine, fomocaine, guafecainol, heptacaine, hexylcaine, hydroxyprocaine, hydroxytetracaine, isobutamben, leucinocaine, levobupivacaine, levoxadrol, lidamidine, lidocaine, lotucaine, menglytate, mepivacaine, meprylcaine, myrtecaine, octacaine, octodrine, oxetacaine, oxybuprocaine, parethoxycaine, paridocaine, phenacaine, piperocaine, piridocaine, polidocanol, pramocaine, prilocaine, procaine, propanocaine, propipocaine, propoxycaine, proxymetacaine, pyrrocaine, quatacaine, quinisocaine, risocaine, rodocaine, ropivacaine, tetracaine, tolycaine, trimecaine, and one of their salts, in particular a hydrochloride, or a mixture thereof. Preferably, the hydrogel according to the invention comprises an anesthetic agent as defined above and in particular lidocaine, mepivacaine or one of their salts such as the hydrochloride.

Examples of antioxidants include, in a non-limiting manner, glutathione, reduced glutathione, ellagic acid, spermine, resveratrol, retinol, L-carnitine, polyols, polyphenols, flavanols, theaflavins, catechins, caffeine, ubiquinol, ubiquinone, alpha-lipoic acid and their derivatives, and a mixture thereof.

Examples of amino acids include, in a non-limiting manner, arginine (e.g., L-arginine), isoleucine (e.g., L-isoleucine), leucine (e.g., L-leucine), lysine (e.g., L-lysine or L-lysine monohydrate), glycine, valine (e.g., L-valine), threonine (e.g., L-threonine), proline (e.g., L proline), methionine, histidine, phenylalanine, tryptophan, cysteine, their derivatives (e.g., N-acetylated derivatives such as N-acetyl-L-cysteine) and a mixture thereof.

Examples of vitamins and their salts include, in a non-limiting manner, vitamins E, A, C, B, especially vitamins B6, B8, B4, B5, B9, B7, B12, and better still pyridoxine and its derivatives and/or salts, preferably pyridoxine hydrochloride.

Examples of minerals include, in a non-limiting manner zinc salts (for example, zinc acetate, especially dehydrated or zinc citrate; preferably zinc citrate will be chosen), magnesium salts, calcium salts (for example, hydroxyapatite, especially in bead form), potassium salts, manganese salts, sodium salts, copper salts (for example, copper sulfate, especially pentahydrate), optionally in hydrated form, and mixtures thereof. Zinc citrate will preferably be chosen as additional component.

Examples of nucleic acids include, in a non-limiting manner, adenosine, cytidine, guanosine, thymidine, cytosine, their derivatives and a mixture thereof. Co-enzymes include coenzyme Q10, CoA, NAD, NADP, and mixtures thereof.

Adrenaline derivatives include adrenaline, noradrenaline and a mixture thereof.

### Extrusion (5)

The hydrogel preparation method may comprise one or more extrusion steps. This extrusion step makes it possible to obtain a more homogeneous hydrogel, in particular with an extrusion force that is as constant as possible, i.e. as regular as possible. For example, the extrusion step can be carried out by means of a sieve whose perforations have a diameter comprised between 50 and 2000 μm. The person skilled in the art knows how to select the perforation diameter according to the mechanical properties of the hydrogel sought.

### Method 2

When the method of the present invention uses method 2, step (1) of preparing the hydrogel comprises the following steps:
(a) preparing a cross-linked polysaccharide from a cross-linking reaction medium comprising one or more polysaccharides, one or more cross-linking agents, a solvent and citrate ions in a quantity sufficient to permit the preparation of a hydrogel comprising a cross-linked polysaccharide and further comprising at least 1 mM citrate ions;
(b) preparing a hydrogel from the cross-linked polysaccharide obtained at the end of step (a) and optionally from a non-cross-linked polysaccharide.

In particular, the cross-linked polysaccharide can be prepared by a method comprising the following steps:
(a1) preparing a cross-linking reaction medium comprising one or more polysaccharides, one or more cross-linking agents, a solvent and citrate ions in a quantity sufficient to permit the preparation of a hydrogel based on a cross-linked polysaccharide comprising at least 1 mM citrate ions; and
(a2) reacting the reaction medium to obtain a cross-linked polysaccharide.

Steps (a), (a1) and (a2) of the method according to method 2 are as described above in the “Cross-linked and/or non-cross-linked polysaccharide” section, with the difference that the reaction medium also comprises citrate ions.

The citrate ions are typically present in the reaction medium in a quantity which makes it possible to obtain a concentration of citrate ions in the hydrogel of at least 1.5 mM, or of at least 2 mM, or of at least 2.5 mM, or of at least 3 mM or of at least 3.5 mM. The maximum concentration of citrate ions in the hydrogel is generally 20 mM or 12 mM. In some embodiments, the quantity of citrate ions present in the reaction medium makes it possible to obtain a concentration of citrate ions in the hydrogel ranging from 2 to 20 mM, or 2 to 12 mM, or from 3 to 11 mM, or from 3 to 9 mM, or from 3 to 8 mM, or from 4 to 8 mM or from 3 to 5 mM.

In some embodiments, the quantity of citrate ions present in the reaction medium makes it possible to obtain a concentration of citrate ions in the hydrogel ranging from 5 to 12 mM.

Citrate ions present in the reaction medium may result from the addition of citric acid or an aqueous solution of citric acid to the reaction medium.

In some embodiments, the citrate ions present in the reaction medium result from the addition of sodium citrate or an aqueous sodium citrate solution to the reaction medium. In some embodiments, the citrate ions present in the reaction medium result from the addition of calcium citrate, or potassium citrate, or magnesium citrate or a solution thereof to the reaction medium.

At the end of step (a2), the cross-linked polysaccharide is typically in the form of a gel comprising citrate ions. This gel is generally directly involved in the remainder of the method of the invention (step (b)). No covalent bonds are formed between polysaccharide and citrate ions.

A hydrogel (step (b)) from the cross-linked polysaccharide obtained at the end of step (a) or (a2) can be prepared conventionally. In particular, the preparation of a hydrogel from the cross-linked polysaccharide obtained at the end of step (a) or (a2) typically comprises one or more of the following conventional steps:
pH adjustment (1);
Dilution (2);
Purification (3);
Addition of at least one additional component (4);
Extrusion (5).

These steps, which are well known to the skilled person, may be as described above in relation to method 1. They may be implemented in the sequential ways described above.

### Hydrogel Sterilization (Step (2))

The method of the present invention comprises a step of sterilizing the prepared hydrogel. Sterilization is preferably carried out by heat, for example in an autoclave.

Sterilization is generally carried out by increasing the temperature of the sterilization medium to a temperature called the plateau temperature, which is maintained for a specified period called the plateau time. Sterilization is preferably carried out at a plateau temperature ranging from 121° C. to 135° C., preferably for a plateau time ranging from 1 minute to 20 minutes with F0>15. The sterilizing value F0 corresponds to the time required, in minutes, at 121° C., to inactivate 90% of the population of microorganisms present in the product to be sterilized. Alternatively, sterilization can be carried out by gamma radiation, UV radiation or by means of ethylene oxide.

The hydrogel obtained at the end of the method according to the invention typically has a pH ranging from 6.8 to 7.8 (physiological pH).

### METHOD 1 OR 2: Optional Step

The method of the present invention (method 1 or 2) may also comprise a step of packaging the hydrogel. The hydrogel is typically packaged in an injection device. The packaging is preferably carried out just before the sterilization step (2). Thus, the sterile hydrogel can be in the form of an injection device pre-filled with the hydrogel, for example a syringe pre-filled with the hydrogel.

### Sterile Hydrogel

The sterile hydrogel obtained by the method of the present invention (method 1 or 2) is a hydrogel based on a cross-linked polysaccharide or a non-cross-linked polysaccharide or a mixture thereof. The sterile hydrogel obtained by the method of the present invention (method 1 or 2) therefore comprises a cross-linked polysaccharide, or a non-cross-linked polysaccharide, or a mixture of a cross-linked polysaccharide and a non-cross-linked polysaccharide. It is understood that the cross-linked polysaccharide may be a mixture of cross-linked polysaccharides.

The sterile hydrogel obtained by the method of the present invention (method 1 or 2) has a physiological pH, i.e., ranging from 6.8 to 7.8. The pH of the sterile hydrogel is preferably greater than or equal to 6.9 and less than or equal to 7.4; 7.3; 7.2; 7.1 or 7. The sterile hydrogel obtained by the method of the present invention (method 1 or 2) and comprising a cross-linked polysaccharide, advantageously has a phase angle õ of less than or equal to 45° at 1 Hz for a deformation of 0.1% or a pressure of 1 Pa, preferably a phase angle δ ranging from 2° to 45° or ranging from 20° to 45°.

The hydrogel obtained by the method of the present invention is preferably an injectable hydrogel, i.e., one which can flow out and be injected manually by means of a syringe equipped with a needle of diameter ranging from 0.1 to 0.5 mm, for example a hypodermic needle of 32 G, 30 G, 27 G, 26 G, 25 G.

The hydrogel obtained by the method of the present invention may comprise from 0.1 to 5% by weight, preferably from 1 to 3% by weight, of polysaccharide (total weight of polysaccharide, i.e., total weight of cross-linked and/or non-cross-linked polysaccharide, for example cross-linked and/or non-cross-linked hyaluronic acid), relative to the total weight of the hydrogel. Thus, when the hydrogel comprises, as the only polysaccharide, a non-cross-linked polysaccharide, the hydrogel obtained by the method of the present invention can therefore comprise from 0.1 to 5% by weight, preferably from 1 to 3% by weight, of non-cross-linked polysaccharide (for example of non-cross-linked hyaluronic acid), relative to the total weight of the hydrogel. When the hydrogel comprises, as sole polysaccharide, a cross-linked polysaccharide, the hydrogel obtained by the method of the present invention can therefore comprise from 0.1 to 5% by weight, preferably from 1 to 3% by weight, of cross-linked polysaccharide (for example cross-linked hyaluronic acid), relative to the total weight of the hydrogel. When the hydrogel comprises the mixture of a cross-linked and non-cross-linked polysaccharide, the hydrogel obtained by the method of the present invention can therefore comprise from 0.1 to 5% by weight, preferably from 1 to 3% by weight, of a mixture of non-cross-linked and cross-linked polysaccharide (for example of non-cross-linked and/or cross-linked hyaluronic acid), relative to the total weight of the hydrogel. In particular, the content of non-cross-linked polysaccharide (for example, hyaluronic acid) may vary from 0.5 to 40% by weight, preferentially from 1 to 40% by weight, more preferentially from 5 to 30% by weight, relative to the total weight of polysaccharide (for example, hyaluronic acid) present in the hydrogel.

The total polysaccharide concentration obtained by the method of the present invention advantageously varies from 1 mg/g to 50 mg/g of hydrogel, more advantageously from 5 mg/g to 35 mg/g of hydrogel, even more advantageously from 10 mg/g to 30 mg/g of hydrogel. Preferably the polysaccharide is hyaluronic acid, even more preferentially sodium hyaluronate.

When the hydrogel comprises a cross-linked polysaccharide, the cross-linked polysaccharide preferably has a molar cross-linking rate of less than or equal to 10%. Preferentially, the hydrogel comprises a cross-linked polysaccharide whose molar degree of cross-linking is greater than 0 and less than or equal to 6%. Even more preferentially, the hydrogel comprises a cross-linked polysaccharide whose molar degree of cross-linking is greater than 0 and less than or equal to 4%. Even more preferably, the hydrogel comprises a cross-linked polysaccharide whose molar degree of cross-linking is greater than 0 and less than or equal to 2%, preferably less than or equal to 1%, even more preferentially less than or equal to 0.8%, especially ranging from 0.1% to 0.5% (number of moles of cross-linking agent(s) per 100 moles of repeating unit of the polysaccharide(s)).

When the hydrogel comprises a cross-linked polysaccharide, the cross-linked polysaccharide preferably has a degree of modification (MOD) of less than or equal to 10%, preferably less than or equal to 6%, preferably less than or equal to 4%, preferably less than or equal to 2%, more preferably less than or equal to 1%. Advantageously, the cross-linked polysaccharide has a degree of modification (MOD) of less than or equal to 1.8%, more preferably less than or equal to 1.5%, preferentially less than or equal to 1.2%, even more preferentially less than 1%.

In some embodiments, the hydrogel comprises an anesthetic agent. The anesthetic agent may be as described above, in particular the anesthetic agent may be mepivacaine, lidocaine or one of their salts; more particularly in the form of a hydrochloride salt; preferably in quantities ranging from 0.1 to 30 mg/ml, for example from 0.5 to 10 mg/ml or more preferentially from 2 to 6 mg/ml.

Sterile hydrogels prepared according to the method of the invention are most particularly useful for filling and/or replacing tissues, in particular soft tissues, especially by injecting the hydrogel into the tissue.

They can be injected using any of the methods known to the person skilled in the art. In particular, they can be administered by means of an injection device suitable for an intra-epidermal and/or intradermal and/or subcutaneous and/or supraperiosteal injection. The injection device may especially be chosen from a syringe, a set of microsyringes, a wire, a laser or hydraulic device, an injection gun, a needleless injection device, or a microneedle roller.

The sterile hydrogels prepared according to the method of the invention are preferably injected subcutaneously.

They can concern deep applications, middle applications and/or superficial applications. They can have therapeutic and/or cosmetic and/or cosmeceutical applications.

In the cosmetic field, hydrogels can be particularly useful for compensating for tissue volume losses due to aging.

They can be used in the prevention and/or cosmetic treatment of an alteration of the surface appearance of the skin. For example, hydrogels can be used in the cosmetic field for preventing and/or treating the alteration of viscoelastic or biomechanical properties of the skin; for filling in volume defects of the skin, especially for filling wrinkles, fine lines and scars; to reduce nasolabial folds and frown lines, to increase the volume of the cheekbones, chin or lips, to restore facial volume, especially the cheeks, temples, contour of the face and area around the eyes; or to reduce the appearance of wrinkles and fine lines.

The method for preparing sterile hydrogels of the present invention is respectful of the properties of hydrogels, i.e., it leads to fewer modifications of the rheological properties of the hydrogels during sterilization. Indeed, a better conservation of the rheological properties of the hydrogels after sterilization (better conservation of the elastic modulus G′, better conservation of the phase angle) was observed compared to hydrogels prepared by a method without addition of citrate ions.

The method of the present invention allows the preparation of sterile hydrogels whose decrease in elastic modulus G′ after sterilization does not exceed 50%, 45%, 40%, 35% or 30% of the value of elastic modulus G′ before sterilization.

Since the addition of citrate ions according to method 1, in particular in the form of a solution, has the effect of slightly diluting the hydrogel, it could be expected that the rheological properties of the hydrogel would be negatively impacted by this addition. Unexpectedly, it was observed that the addition of a solution containing citrate ions had a favorable effect on the hydrogel properties during sterilization.

Furthermore, the addition of citrate ions, especially in the form of a solution, preserves the properties of the hydrogel over time. Indeed, a better conservation of the rheological properties of the hydrogels over time (better conservation of the elastic modulus G′, better conservation of the phase angle) has been observed compared to hydrogels prepared by a method without addition of citrate ions that tend to have their rheological properties decrease more consistently over the course of months.

The use of citrate ions, in particular in the form of a solution, in a method for preparing a hydrogel thus makes it possible to protect a hydrogel comprising a cross-linked and/or non-cross-linked polysaccharide, in particular comprising at least one cross-linked polysaccharide, from the degradation of its rheological properties during sterilization, preferably by heat. The use of citrate ions, in particular in the form of a solution, in a method for preparing a hydrogel comprising a cross-linked and/or non-cross-linked polysaccharide, in particular comprising at least one cross-linked polysaccharide, also makes it possible to preserve the length of the cross-linked and/or non-cross-linked polysaccharide chains.

The use of citrate ions, in particular in the form of a solution, in a method for the preparation of a hydrogel comprising a cross-linked and/or non-cross-linked polysaccharide, in particular comprising at least one cross-linked polysaccharide, also makes it possible to preserve the stability of the hydrogels, especially after sterilization, over time, in particular to increase the stability of hydrogels, especially after sterilization, in comparison with identical hydrogels not comprising citrate ions. In other words, the hydrogels obtained according to the invention maintain their rheological properties more effectively over time after sterilization.

Moreover, it is known that the additional presence of an anesthetic agent in a hydrogel comprising a cross-linked and/or non-cross-linked polysaccharide leads to an increased degradation of the rheological properties of the hydrogels during sterilization, preferably by heat. The addition of citrate ions limits these effects. The hydrogels obtained by the method of the present invention comprising an anesthetic agent exhibit lesser degradation of their rheological properties after sterilization compared to hydrogels comprising an anesthetic agent prepared by an equivalent method without addition of citrate ions.

It should also be observed that a method for the preparation of a hydrogel according to method 2, in which step (a2) is carried out at a pressure P less than or equal to atmospheric pressure and at a temperature T greater than the eutectic point temperature of the reaction medium as measured at pressure P and less than the freezing point temperature of the reaction medium as measured at pressure P, leads to very good preservation of the rheological properties of the hydrogels, since the hydrogels prepared under these conditions may be more sensitive to sterilization.

The examples which follow are given for illustrative purposes but should in no way be considered as limiting the present invention.

### EXAMPLES

### 1. Materials

Non-cross-linked sodium hyaluronate
BDDE (Sigma Aldrich)
0.25 M NaOH
1 M HCl
Citric acid (Sigma Aldrich) (CAS No: 5949-29-1)
Divinylsulfone (Sigma Aldrich)
Phosphate buffer (BBraun),
Lidocaine hydrochloride
Three-dimensional stirrer
DHR-2 rheometer
Dynamometer and test bench
Paddle mill homogenizer
Sterile polyethylene bag

### 2. Methods

### Measuring the Viscoelastic Properties

The viscoelastic properties of the hydrogels obtained were measured using a rheometer (DHR-2) having a stainless steel cone (1°−40 mm) with cone-plane geometry and an anodized aluminum Peltier plate (42 mm) (air gap 24 μm).

0.5 g of sterilized hydrogel is deposited between the Peltier plate and said cone. Then a strain sweep is carried out at 1 Hz and 25° C. The elastic modulus G′, the viscous modulus G″ and the phase angle δ are reported for a stress of 5 Pa. The measurements are carried out in the LVER linear domain.

The stress at the intersection of G′ and G″, T, is determined at the intersection of the curves of the G′ and G″ moduli and is expressed in Pascals.

### 3. EXAMPLES

### 3.1 Example 1

Two hydrogels of cross-linked hyaluronic acid are prepared from a high molecular weight 3 MDa hyaluronic acid and BDDE in a 0.25 M aqueous sodium hydroxide solution (cross-linking for 1 month at −20° C.). The cross-linked polysaccharides have a molar degree of cross-linking of 0.2%. PBS phosphate buffer and 1 N HCl solution are then added to the cross-linked polysaccharides until a pH of 7.3+0.5 is obtained. The hydrogels obtained are homogenized using a three-dimensional stirrer. The mixtures are dialyzed. The hydrogels obtained have either a concentration of 15 mg of hyaluronic acid per gram of product (hydrogel A) or a concentration of 23 mg of hyaluronic acid per gram of product (hydrogel B).

A solution of non-cross-linked sodium hyaluronate of high molecular weight is then added to the hydrogels obtained as lubricant (same quantity of sodium hyaluronate of high molecular weight in the various mixtures) comprising, or not comprising, citrate ions.

The solution comprising citrate ions and high molecular weight sodium hyaluronate is prepared as follows. The citric acid (in powder form) is dissolved in the phosphate buffer, and the pH is then adjusted with 5 M NaOH to reach a physiological pH (pH=6.8-7.8); finally the high molecular weight sodium hyaluronate is added as a lubricant. The concentration of citrate ions in the solution is adjusted taking into account the dilution effect following the addition of this solution to the hydrogel based on cross-linked hyaluronic acid. Indeed, the citrate ion concentration shown in Table 1 corresponds to the final concentration in the hydrogel.

The prepared solution comprising citric acid and high molecular weight sodium hyaluronate or the solution comprising high molecular weight sodium hyaluronate alone is then mixed with the cross-linked hyaluronic acid based hydrogel in a stirring tank.

The products obtained (hydrogels A, B) were sieved and then packaged in a syringe. Finally, the products were sterilized by autoclave (plateau temperature comprised between 121° C. and 135° C. with F0≥15).

Before and after sterilization, the prototypes were analyzed. The elastic modulus G′ and the phase angle δ were determined. The results are presented in Table 1 below.

The prototypes have a molar cross-linking rate of 0.2%.

TABLE 1

| | Hydrogels | | | |
|---|---|---|---|---|
| | A | | B | |
| | A1 | A2 | B1 | B2 |
| Molar concentration of citrate ions | 0 | 3.06 | 0 | 3.06 |
| (mM) in the final hydrogel | | | | |
| Mass percentage of citrate ions | 0 | 0.06% | 0 | 0.06% |
| relative to the mass of the final | | | | |
| hydrogel* | | | | |
| G′ (1 Hz) before sterilization | 261.7 | 245.0 | 511.1 | 499.2 |
| δ before sterilization | 15.4 | 15.8 | 13.3 | 13.4 |
| ΔG′ (%)¹ | −54.5 | −33.2 | −37.9 | −18.8 |
| Δ δ (%)² | 67.5 | 39.3 | 58.9 | 36.2 |

¹ΔG′ (%) = (G′ after sterilization − G′ before sterilization)/(G′ before sterilization) *100
²Δ δ (%) = (δ after sterilization − δ before sterilization)/(δ before sterilization) *100
*1 mL of hydrogel was considered to weigh one gram.

It is observed that the hydrogels prepared from a method according to the invention comprising a step of addition of a solution of citric acid (hydrogels A2, B2) exhibit lesser degradation of their rheological properties after sterilization compared with hydrogels prepared by an equivalent method without addition of a solution of citric acid (hydrogels A1, B1). Indeed, hydrogels A2 and B2 have been observed to have a higher elastic modulus (G′) after sterilization than hydrogels A1 and B1 after sterilization. The decrease in elastic modulus (G′) is therefore lower after sterilization for hydrogels A2 and B2. It has also been observed that hydrogels A2 and B2 have a lower phase angle (δ) after sterilization than hydrogels A1 and B1 after sterilization.

### 3.2 Example 2

A hydrogel of cross-linked hyaluronic acid is prepared from a high molecular weight 4 MDa hyaluronic acid and BDDE in a 0.25 M aqueous sodium hydroxide solution. The cross-linked polysaccharide has a molar degree of cross-linking of 2%. PBS phosphate buffer and 1 N HCl solution are then added to the cross-linked polysaccharides until a pH of 7.3±0.5 is obtained. The hydrogel obtained is homogenized using a three-dimensional stirrer. The mixture is dialyzed. The hydrogels obtained have a concentration of 15 mg of hyaluronic acid per gram of product.

The following are then added to the resulting hydrogels, as appropriate:
a solution of sodium hyaluronate of high molecular weight as lubricant (same quantity in the various mixtures) with or without citrate ions;
an aqueous solution of lidocaine hydrochloride to obtain 0.3% by weight of lidocaine hydrochloride relative to the weight of the final hydrogel;
citric acid solution.

### Hydrogels C

For the hydrogels C, only a non-cross-linked sodium hyaluronate solution of high molecular weight with or without citrate ions is added.

The solution comprising citrate ions and high molecular weight sodium hyaluronate is prepared as follows. The citric acid (in powder form) is dissolved in the phosphate buffer, the pH is then adjusted with 5 M NaOH to reach a physiological pH (pH=6.8-7.6), and, finally, the high molecular weight sodium hyaluronate is added as lubricant. The concentration of citrate ions in the solution is adjusted taking into account the dilution effect following the addition of this solution to the mixture comprising the cross-linked hyaluronic acid. Indeed, the citrate ion concentration shown in Table 2 corresponds to the final concentration in the hydrogel.

The prepared solution comprising citric acid and high molecular weight sodium hyaluronate is then mixed with the mixture comprising cross-linked hyaluronic acid in a stirring tank.

### Hydrogels D

For hydrogels D, a high molecular weight sodium hyaluronate solution, an anesthetic solution and, optionally, a citric acid solution are added.

The citric acid solution is added at the same time as the anesthetic solution, the citric acid solution and anesthetic solution being added after addition of the high molecular weight sodium hyaluronate solution.

A citric acid solution is prepared. Citric acid (in powder form) is first dissolved in phosphate buffer and then 5 M NaOH is added to adjust the pH to a physiological level. The objective is to make a solution that is 100 times more concentrated than the actual concentration desired in the final hydrogel. This avoids an excessive dilution effect of the hydrogel due to the addition of the citric acid solution.

The products obtained (hydrogels C and D) were sieved through around one micron and then packaged in a syringe.

Finally, the products were sterilized by autoclave (plateau temperature comprised between 121° C. and 135° C. with F0≥15).

5 Before and after sterilization, the prototypes were analyzed. The elastic modulus G′ and the phase angle δ were determined. The results are presented in Table 2 below.

TABLE 2

| | Hydrogels | | | | | | | |
|---|---|---|---|---|---|---|---|---|
| | C | | D | | | | | |
| | C1 | C2 | D1 | D2 | D3 | D4 | D5 | D6 |
| Molar | 0 | 3.06 | 0 | 1.53 | 4.59 | 7.65 | 10.71 | 12.24 |
| concentration | | | | | | | | |
| of citrate ions | | | | | | | | |
| (mM) | | | | | | | | |
| in the final | | | | | | | | |
| hydrogel | | | | | | | | |
| Mass | 0 | 0.06% | 0% | 0.03% | 0.09% | 0.14% | 0.20% | 0.23% |
| percentage of | | | | | | | | |
| citrate ions in | | | | | | | | |
| relation to the | | | | | | | | |
| total mass of | | | | | | | | |
| the final | | | | | | | | |
| hydrogel* | | | | | | | | |
| G′ (1 Hz) | 130.1 | 127.8 | 124.9 | 122.7 | 120.9 | 122.1 | 122.3 | 121.1 |
| before | | | | | | | | |
| sterilization | | | | | | | | |
| δ before | 13 | 15.3 | 13.5 | 13.5 | 13.5 | 13.4 | 13.3 | 13.4 |
| sterilization | | | | | | | | |
| ΔG′ (%)¹ | −18.9 | −10.7 | −33.0 | −29.4 | −23.4 | −18.2 | −14.4 | −16.6 |
| Δ δ (%)² | 31.6 | 8.8 | 45.5 | 36.3 | 27.7 | 21.1 | 21.1 | 22.1 |

¹ΔG′ (%) = (G′ after sterilization − G′ before sterilization)/(G′ before sterilization) *100
²Δ δ (%) = (δ after sterilization − δ before sterilization)/(δ before sterilization) *100
*1 mL of hydrogel was considered to weigh one gram.

It is observed that the hydrogels prepared from a method according to the invention comprising a step of addition of citrate ions (addition of a non-cross-linked high molecular weight sodium hyaluronate solution comprising citrate ions or addition of a citric acid solution) (hydrogels C2, D2 to D6) exhibit fewer changes of their rheological properties after sterilization compared with hydrogels prepared by an equivalent method without addition of citrate ions (hydrogels C1 and D1). Indeed, it was observed that hydrogels C2 and D2 to D6 have a higher elastic modulus (G′) after sterilization than hydrogels C1 and D1. The decrease in elastic modulus (G′) is therefore lower after sterilization for hydrogels C2 and D2 to D6. It has also been observed that hydrogels C2 and D2 to D6 have a lower phase angle (δ) after sterilization than hydrogels C1 and D1.

After 1 month at 40° C., hydrogel C2 prepared using a method according to the invention comprising a step of addition of a solution comprising citrate ions does not show any changes in its rheological properties compared with hydrogel C1 prepared by an equivalent method without addition of such a solution.

### 3.3 Example 3

A hydrogel of cross-linked hyaluronic acid is prepared from a high molecular weight 4 MDa hyaluronic acid and BDDE in a 0.25 M aqueous sodium hydroxide solution. The cross-linked polysaccharide has a degree of cross-linking of 2%. PBS phosphate buffer and 1 N HCl solution are then added to the cross-linked polysaccharides until a pH of 7.3±0.5 is obtained. The hydrogel obtained is homogenized using a three-dimensional stirrer. The mixture is dialyzed. The hydrogels obtained have a concentration of 15 mg of hyaluronic acid per gram of product

To the hydrogels obtained are then added:
a solution of high molecular weight sodium hyaluronate as lubricant (same quantity in different mixtures);
an aqueous solution of lidocaine hydrochloride to obtain 0.3% by weight of lidocaine hydrochloride relative to the weight of the final hydrogel;
optionally, a citric acid solution.

For hydrogels E2 and E3, the citric acid solution is added at the same time as the anesthetic solution, the citric acid solution and anesthetic solution being added after addition of the high molecular weight sodium hyaluronate solution.

A citric acid solution is prepared. Citric acid (in powder form) is first dissolved in phosphate buffer and then 5 M NaOH is added to adjust the pH to a physiological level. The objective is to make a solution that is 100 times more concentrated than the actual concentration desired in the final hydrogel. This avoids an excessive dilution effect of the hydrogel due to the addition of the citric acid solution.

The products obtained (hydrogels E1, E2 and E3) were sieved through around one micron and then packaged in a syringe.

Finally, the products were sterilized by autoclave (plateau temperature comprised between 121° C. and 135° C. with F0≥15).

Before and after sterilization, the hydrogels E1-E3 were analyzed. The elastic modulus G′ and the phase angle δ were determined. The results are presented in Table 3 below.

TABLE 3

| Hydrogels | E1 | E2 | E3 |
|---|---|---|---|
| Molar concentration of citrate | 0 | 3.06 | 7.65 |
| ions (mM) in the final hydrogel | | | |
| G′ (1 Hz) before sterilization | 127.5 | 130.2 | 123.9 |
| G′ (1 Hz) at T0 months (after | 70.3 | 90.0 | 103.9 |
| sterilization) | | | |
| G′ (1 Hz) at T2 months (after | 61.0 | 86.9 | 99.8 |
| sterilization) | | | |
| ΔG′ (%)¹ | −13.2 | −3.4 | −3.9 |

¹ΔG′ (%) = (G′ T 2 months − G′ T0)/(G′ T0) *100

After 2 months at 40° C., hydrogels E2 and E3 prepared using a method according to the invention comprising a step of addition of a solution comprising citrate ions show a minor change of their rheological properties compared with hydrogel E1 prepared by an equivalent method without addition of such a solution.

### 3.4 Example 4

A hydrogel is prepared from a 1.5 MDa high molecular weight hyaluronic acid and divinyl sulfone in a 0.25 M aqueous sodium hydroxide solution mixed beforehand with 0.1 M sodium citrate (cross-linking for 4 hours at 21° C.). The cross-linked polysaccharide has a degree of cross-linking of 0.5%. Phosphate buffer and 1 N HCl solution are then added to the cross-linked polysaccharides until a pH of 7.3±0.5 is obtained. The hydrogel obtained is homogenized using a three-dimensional stirrer. The hydrogel obtained has a concentration of 23 mg of hyaluronic acid per gram of product (hydrogel F1).

A solution of sodium hyaluronate of high molecular weight is then added to the hydrogel obtained as a lubricant;

The hydrogel obtained was sieved and then packaged in a syringe.

Finally, the hydrogel obtained was sterilized by autoclave (plateau temperature comprised between 121° C. and 135° C. with F0≥15).

After sterilization, the hydrogel F1 was analyzed. The elastic modulus G′ and the phase angle δ were determined. The results are presented in Table 4 below.

TABLE 4

| Hydrogels | F1 |
|---|---|
| Molar concentration of citrate ions (mM) | 20 |
| in the final hydrogel | |
| Molar concentration (mM) of the sodium | 100.25 |
| citrate added to the cross-linking | |
| reaction medium | |
| Mass percentage of citrate ions relative | 0.38% |
| to the mass of the final hydrogel* | |
| G′ (1 Hz) before sterilization | 346 |
| δ before sterilization | 17.1 |
| ΔG′ (%)¹ | −16.4 |
| Δ δ (%)² | 11.3 |

¹ΔG′ (%) = (G′ after sterilization − G′ before sterilization)/(G′ before sterilization) *100
²Δ δ (%) = (δ after sterilization − δ before sterilization)/(δ before sterilization) *100
*1 mL of hydrogel was considered to weigh one gram.

It is observed that the hydrogel prepared from a method according to the invention has good rheological properties.

### 3.5 Example 5

Three hydrogels are prepared from a high molecular weight 1.5 MDa hyaluronic acid and BDDE in a 0.25 M aqueous sodium hydroxide solution (cross-linking for 72 hours at 21° C.).

For the hydrogels G2 and G3, 0.1 M sodium citrate was mixed beforehand with the 0.25 M aqueous sodium hydroxide solution. The cross-linked polysaccharide has a degree of cross-linking of 2.3%. Phosphate buffer and 1 N HCl solution are then added to the cross-linked polysaccharides until a pH of 7.3±0.5 is obtained. The hydrogels obtained are homogenized using a three-dimensional stirrer. The hydrogels obtained have a concentration of 23 mg of hyaluronic acid per gram of product (hydrogel G1, hydrogel G2 and hydrogel G3).

A solution of sodium hyaluronate of high molecular weight is then added as lubricant to the hydrogels obtained (same quantity in the various mixtures).

The hydrogels obtained were sieved and then packaged in a syringe.

Finally, the hydrogels obtained were sterilized by autoclave (plateau temperature comprised between 121° C. and 135° C. with F0≥15).

After sterilization, the hydrogels G1, G2 and G3 were analyzed. The elastic modulus G′ and the phase angle δ were determined. The results are presented in Table 5 below.

TABLE 5

| Hydrogel | G1 | G2 | G3 |
|---|---|---|---|
| Molar concentration | 0.00 | 25.00 | 100.25 |
| (mM) of the sodium | | | |
| citrate added to the | | | |
| cross-linking reaction | | | |
| medium | | | |
| Molar concentration of | 0.0 | 5.0 | 20.0 |
| citrate ions (mM) in | | | |
| the final hydrogel | | | |
| Mass percentage of | 0.0 | 0.09% | 0.38% |
| sodium citrate relative | | | |
| to the mass of the final | | | |
| hydrogel* | | | |
| G′ (1 Hz) before | 193.0 | 199.6 | 194.1 |
| sterilization | | | |
| δ before sterilization | 14.3 | 12.2 | 14.4 |
| ΔG′ (%)¹ | −26.9 | −13.3 | −19.6 |
| Δ δ (%)² | 30.3 | 13.9 | 14.2 |

¹ΔG′ (%) = (G′ after sterilization − G′ before sterilization)/(G′ before sterilization) *100
²Δ δ (%) = (δ after sterilization − δ before sterilization)/(δ before sterilization) *100
*1 mL of hydrogel was considered to weigh one gram.

It is observed that hydrogels prepared from a method according to the invention comprising citrate ions in the cross-linking reaction medium exhibit fewer changes in their rheological properties after sterilization compared with hydrogels prepared by an equivalent method without addition of citrate ions.

1. A method for preparing a sterile hydrogel comprising a cross-linked polysaccharide, a non-cross-linked polysaccharide, or a mixture thereof, the method comprising the following steps:
(1) preparing a hydrogel comprising a cross-linked polysaccharide, a non-cross-linked polysaccharide or mixture thereof and further comprising at least 1 mM citrate ions; and
(2) sterilizing the hydrogel comprising at least 1 mM citrate ions to obtain a sterile hydrogel comprising a cross-linked polysaccharide, a non-cross-linked polysaccharide or a mixture thereof.

2. The method according to claim 1, wherein step (1) comprises the addition, to the cross-linked polysaccharide or to the non-cross-linked polysaccharide or to their mixture, of a solution comprising citrate ions in a quantity sufficient to obtain a citrate ion concentration of at least 1 mM in the hydrogel.

3. The method according to claim 1, wherein step (1) comprises the addition, to the cross-linked polysaccharide or to the non-cross-linked polysaccharide or to their mixture, of citrate ions in the powder form in a quantity sufficient to obtain a citrate ion concentration of at least 1 mM in the hydrogel.

4. The method according to claim 2 wherein the solution comprising citrate ions has a pH ranging from 6.8 to 7.8.

5. The method according to claim 2 wherein the solution comprising citrate ions is a solution of citric acid or sodium citrate.

6. The method according to claim 17, wherein the physiologically acceptable buffer is a phosphate buffer.

7. The method according to claim 2 wherein the quantity of citrate ions added to the hydrogel makes it possible to obtain a citrate ion concentration ranging from 1 to 12 mM in the hydrogel.

8. The method according to claim 2 wherein the step (1) of preparing a hydrogel comprises one or more of the following conventional steps:
pH adjustment;
Dilution;
Purification;
Addition of at least one additional component;
Extrusion.

9. The method according to claim 8 comprising a dilution step and/or a step of adding at least one additional component, wherein citrate ions are added in the dilution step and/or in the step of adding at least one additional component.

10. The method according to claim 1 wherein step (1) comprises the following steps:
(a) preparing a cross-linked polysaccharide from a cross-linking reaction medium comprising one or more polysaccharides, one or more cross-linking agents, a solvent and citrate ions in a quantity sufficient to permit the preparation of a hydrogel based on a cross-linked polysaccharide further comprising at least 1 mM citrate ions;
(b) preparing a hydrogel from the cross-linked polysaccharide obtained at the end of step (a).

11. The method according to claim 1 wherein the polysaccharide is a hyaluronic acid.

12. The method according to claim 1 further comprising a step of packaging the hydrogel after step (1) and before step (2).

13. The method according to claim 1 wherein the sterilization is heat sterilization.

14. A sterile hydrogel comprising a cross-linked polysaccharide, a non-cross-linked polysaccharide or a mixture thereof, obtained by the method according to claim 1.

15. A method for protecting a hydrogel comprising a cross-linked polysaccharide, a non-cross-linked polysaccharide or a mixture thereof from degradation of its rheological properties during sterilization comprising the addition of citrate ions in the hydrogel comprising a cross-linked polysaccharide, a non-cross-linked polysaccharide or a mixture thereof.

16. A method for preserving the stability over time of a hydrogel comprising a cross-linked polysaccharide, a non-cross-linked polysaccharide or a mixture thereof comprising the addition of citrate ions in the hydrogel comprising a cross-linked polysaccharide, a non-cross-linked polysaccharide or a mixture thereof.

17. The method according to claim 2 wherein the solution comprising citrate ions is a solution of citric acid or sodium citrate in a physiologically acceptable buffer.

18. A method for filling and/or replacing tissues comprising the injection of the sterile hydrogel of claim 14 into the tissue.

19. A cosmetic method for preventing and/or treating the alteration of viscoelastic or biomechanical properties of the skin; for filling in volume defects of the skin; to reduce nasolabial folds and frown lines, to increase the volume of the cheekbones, chin or lips, to restore facial volume; or to reduce the appearance of wrinkles and fine lines comprising administering to a subject the sterile hydrogel of claim 14.
